(19) United States
(12) Patent Application Publication
(10) Pub. No.: US 2013/0281945 A1
(43) Pub. Date: Oct. 24, 2013

(54) Clean Seat Undie Protector

(71) Applicant: Francine Gallo, Brooklyn, NY (US)

(72) Inventor: Francine Gallo, Brooklyn, NY (US)

(21) Appl. No.: 13/851,071

(22) Filed: Mar. 26, 2013

Related U.S. Application Data
(60) Provisional application No. 61/685,871, filed on Mar. 26, 2012.

(51) Int. Cl. A61F 13/53

(52) U.S. Cl. CPC A61F 13/53
USPC 604/312; 604/377; 604/289

(57) ABSTRACT

The invention provides a sanitary pad that protects and promotes hygiene. The Clean Seat Undie Protector is an improved sanitary pad for dealing with incontinence issues. The Clean Seat comes in different sizes to accommodate the various needs of the consumer and to allow for different types of female and male underwear. The present invention adhesively attaches to the inside of one's underwear. The Clean Seat pad is fabricated with a cotton absorbable material to contain accidental leaks or discharges. The absorbent pad comprises a water resistant sheet with a peel off adhesive strip and cover on one side.

### CLAIM OF PRIORITY

This patent application claims priority under 35 USC 119 (e) (1) from U.S Provisional Patent Application Ser. Number 61/685,871 filed Mar. 26, 2012, of common inventorship herewith entitled, “Clean Seat Undie Protector.”

### FIELD OF THE INVENTION

The present invention pertains to the field of hygiene products, and more specifically to the field of sanitary pads.

### BACKGROUND OF THE INVENTION

The prior art has put forth several designs for sanitary pads or shields. Among these are:

U.S. Patent Design 275,232 to Barbara J. Jones describes an ornamental design of a female sanitary napkin.

U.S. Patent Design 402,756 to Kamela J. Darby describes an ornamental design for a garment shield.

U.S. Pat. No. 7,195,619 to Francis J. Manasek describes an absorbent article for protection against anal leakage. This anal region panty liner has an impervious outer layer, an absorbent middle layer and a liquid permeable body layer. The panty liner is held in place against an undergarment by removable adhesive. It absorbs anal leakage and fits both male and female anatomy. A unique feature of one embodiment is a pear like shape having a wide area covering the buttocks and a narrow part extending forward between the legs, terminating short of the genital area and comfortable for both males and females. An alternate female version continues forward to cover the vaginal or urethral region. The preferred embodiment is thicker in the crotch area, having an absorbent pad situated retro scrotally along the center and an additional inter buttock absorbent ridge. An alternate embodiment has an absorbent dam situated retro scrotally. Another alternate embodiment is flat, being of uniform thickness.

None of these prior art references describe the present invention.

### SUMMARY OF THE INVENTION

It is an object of the present invention to provide a unisex sanitary pad for garment protection from urinary and fecal incontinence.

### BRIEF DESCRIPTION OF THE DRAWINGS

FIG. 1 is an illustrative functional top view showing a disposable underwear shield inserted in a pair of men's briefs.

FIG. 2 is an illustrative functional top view showing a disposable underwear shield inserted in a pair of women's panties.

FIG. 3 is an illustrative functional top view showing a disposable underwear shield inserted in a pair of men's boxer shorts.

FIG. 4 is an illustrative diagonal view showing a quilted cotton cover or top and an adhesive backing with a removable peel off paper strip.

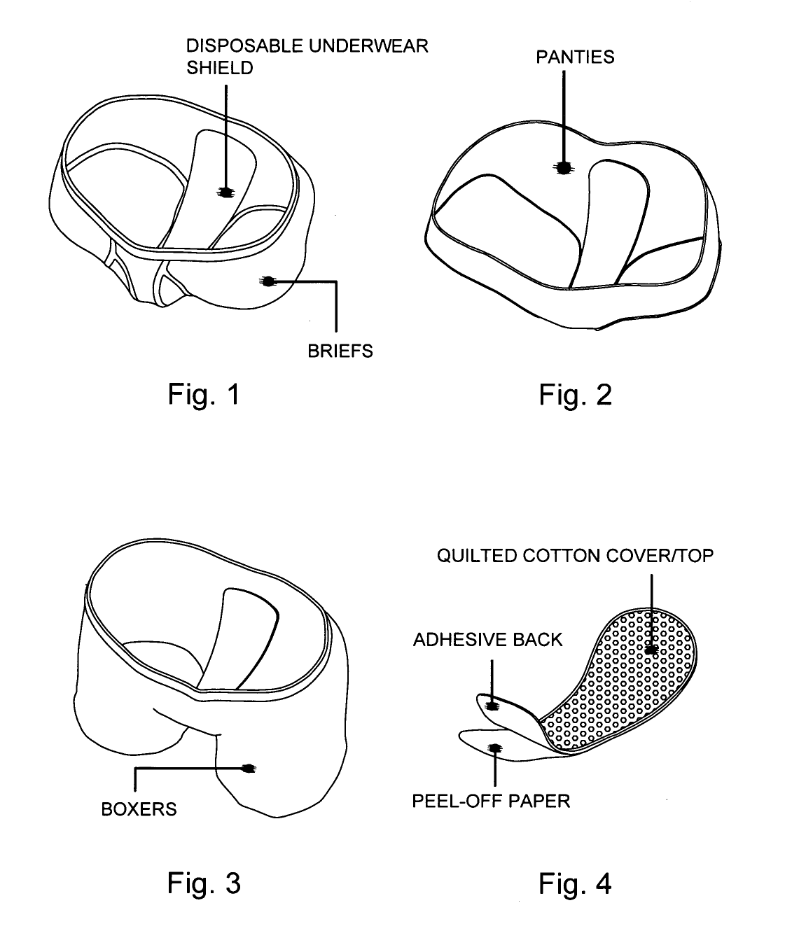

### DETAILED DESCRIPTION OF THE INVENTION

Many contributing factors exist for urinary and fecal incontinence or leakage in women and men. The most common reasons for leakage or complete incontinence in women are an over active bladder or OAB, stress leakage of small amounts of urine during physical movement such as exercise or sneezing, weight gain, pregnancy and childbirth. Female incontinence has much to do with the way women's bodies are designed. Pregnancy and childbirth weaken or strain the muscles that control the bladder. A decrease in estrogen during a woman's menopausal years effects bladder control and causes incontinence. Men suffer from urinary and fecal incontinence after radiation therapy for prostate conditions and lower bowel cancers. Although incontinence may occur at any age, it is most common among the elderly population.

The present invention, hereinafter referred to as the Clean Seat or the Clean Seat Undie Protector, is a sanitary pad that protects and promotes hygiene. The Clean Seat Undie Protector is an improved sanitary pad for dealing with incontinence issues. The Clean Seat comes in different sizes to accommodate the various needs of the consumer and to allow for different types of female and male underwear. The present invention adhesively attaches to the inside of one's underwear. The Clean Seat pad is fabricated with a cotton absorbable material 22 to contain accidental leaks or discharges. The absorbent pad comprises a water resistant sheet 24 with a peel off adhesive strip 28 and cover 26 on one side. The water resistant sheet comprises water resistant paper, or a thin layer of plastic. The adhesive strip attaches to a user's underwear for a close fit to the middle of the underwear. The standard dimensions of these oblong oval pads are approximately eleven inches in length by four inches in width. The pads are modifiable in size for a user's gender and the type of undergarment a user wears. An alternate embodiment is a smaller and rounder sanitary pad used by young men and women involved in sport or training exercises. This embodiment soaks up sweat and masks odors with an added scent such as pine.

An alternative embodiment is designed for an adult male user. In this embodiment, the pad is elongate and is designed to fully cover the area of the anus, to prevent leakage.

An additional alternative embodiment includes the pad being treated with medicated cream, or hemorrhoid cream.

All the Clean Seat products avoid embarrassment from leakage showing through one's garments and also protect one's underwear from stain and frequent replacement. The Clean Seat is a disposable underwear shield that obviates unsanitary conditions caused by hemorrhoids, improper hygiene, and sweat, or leakage from the bladder. The Clean Seat product line has different sizes for men and women, and different varieties of the pads such as hypoallergenic or infused with hemorrhoid cream. The present invention is easy to use, comfortable and discreet, and sized to comfortably fit all users who require a sanitary product. The Clean Seat stores easily in one's purse or pocket, so a user always has a spare Clean Seat available. Affordably priced, the Clean Seat Undie Protector will be received enthusiastically by consumers of all ages.

Although this invention has been described with respect to specific embodiments, it is not intended to be limited thereto and various modifications which will become apparent to the person of ordinary skill in the art are intended to fall within the spirit and scope of the invention as described herein taken in conjunction with the accompanying drawings and the appended claims.

1. A unisex sanitary pad device for garment protection from urinary and fecal incontinence, comprising: a pad adhesively attaches to the inside of one's underwear, fabricated with a cotton absorbable material to contain accidental leaks or discharges, further comprising a water resistant sheet having a peel off adhesive strip and cover on one side of the absorbent pad, wherein the adhesive is on the outer side of the pad to secure the pad to the underwear of the user and the adhesive is attached to the water resistant sheet.

2. The sanitary pad device of claim 1 wherein the pad is a smaller and rounder sanitary pad used by young men and women involved in sport or training exercises, wherein the pad soaks up sweat and masks odors with an added scent.

3. The sanitary pad device of claim 2 wherein the scent is pine.

4. The sanitary pad device of claim 1 designed for an adult male user, wherein the pad is elongate and is designed to fully cover the area of the anus, to prevent leakage.

5. The sanitary pad device of claim 1 wherein the pad is treated with medicated cream, or hemorrhoid cream.
